(19) United States
(12) Patent Application Publication
(10) Pub. No.: US 2025/0298001 A1
(43) Pub. Date: Sep. 25, 2025

(54) DETECTION DEVICE

(71) Applicant: Japan Display Inc., Tokyo (JP)

(72) Inventors: Akihiko FUJISAWA, Tokyo (JP); Kaoru Ito, Tokyo (JP); Daichi Abe, Tokyo (JP)

(21) Appl. No.: 19/085,520

(22) Filed: Mar. 20, 2025

(30) Foreign Application Priority Data
Mar. 22, 2024 (JP) 2024-047175

(51) Int. Cl. G01N 33/483

(52) U.S. Cl. CPC G01N 33/4833

(57) ABSTRACT

According to an aspect, a detection device includes: an optical sensor comprising photodiodes; a light source including light-emitting elements; and an object placement member that has a light-transmitting property and is configured to be disposed between the optical sensor and the light source, and on which objects to be detected are to be placed. The photodiodes are arranged in a matrix having a row-column configuration and are configured to be sequentially driven along the second direction at least one row by one row. The light-emitting elements are arranged in a matrix having a row-column configuration and are configured to be sequentially driven along the second direction at least one row by one row. At a given time, photodiodes to be driven among the photodiodes correspond to light-emitting elements to be lit among the light-emitting elements in plan view.

### CROSS-REFERENCE TO RELATED APPLICATION

This application claims the benefit of priority from Japanese Patent Application No. 2024-047175 filed on Mar. 22, 2024, the entire contents of which are incorporated herein by reference.

### BACKGROUND

### 1. Technical Field

What is disclosed herein relates to a detection device.

### 2. Description of the Related Art

Japanese Patent Application Laid-open Publication No. 2018-033430 (JP-A-2018-033430) discloses an image acquisition device that includes an optical sensor, a container to contain microorganisms and a culture medium, and a light source, and acquires, over time, images indicating growth of the microorganisms in the container. In JP-A-2018-033430, one point light source is disposed for a plurality of microorganisms (objects to be detected) in a culture vessel.

Such a detection device is required to detect the objects to be detected in a detection area having a larger area, and thus, requires a plurality of light-emitting elements. In this case, the multiple light-emitting elements are continuously lit in the entire area for a duration of scanning the optical sensor (photosensor in JP-A-2018-033430) along predetermined directions. Thus, the light-emitting elements emit light also to areas of the optical sensor that are not being driven, and therefore, may cause a power loss.

For the foregoing reasons, there is a need for a detection device capable of reducing the power loss.

### SUMMARY

According to an aspect, a detection device includes: an optical sensor comprising a plurality of photodiodes arranged in a planar configuration; a light source including a plurality of light-emitting elements configured to emit light to the photodiodes; and an object placement member that has a light-transmitting property and is configured to be disposed between the optical sensor and the light source, and on which a plurality of objects to be detected are to be placed. The photodiodes are arranged in a matrix having a row-column configuration in a first direction and a second direction intersecting the first direction and are configured to be sequentially driven along the second direction at least one row by one row. The light-emitting elements are arranged in a matrix having a row-column configuration in the first direction and the second direction and are configured to be sequentially driven along the second direction at least one row by one row. At a given time, photodiodes to be driven among the photodiodes correspond to light-emitting elements to be lit among the light-emitting elements in plan view.

### BRIEF DESCRIPTION OF THE DRAWINGS

FIG. 1 is a sectional view schematically illustrating a detection device according to an embodiment;

FIG. 2 is a block diagram illustrating a configuration example of the detection device according to the embodiment;

FIG. 3 is a circuit diagram illustrating an optical sensor of the detection device according to the embodiment;

FIG. 4 is a circuit diagram illustrating a configuration example of a sensor pixel and a detection circuit of the detection device according to the embodiment;

FIG. 5 is a timing waveform diagram illustrating an operation example of the detection device according to the embodiment;

FIG. 6 is a plan view schematically illustrating the operation example of the detection device according to the embodiment; and

FIG. 7 is a sectional view schematically illustrating the operation example of the detection device according to the embodiment.

### DETAILED DESCRIPTION

The following describes a mode (embodiment) for carrying out the present disclosure in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present disclosure, in expressing an aspect of disposing another structure on or above a certain structure, a case of simply expressing “on” includes both a case of disposing the other structure immediately on the certain structure so as to contact the certain structure and a case of disposing the other structure above the certain structure with still another structure interposed therebetween, unless otherwise specified.

### Embodiment

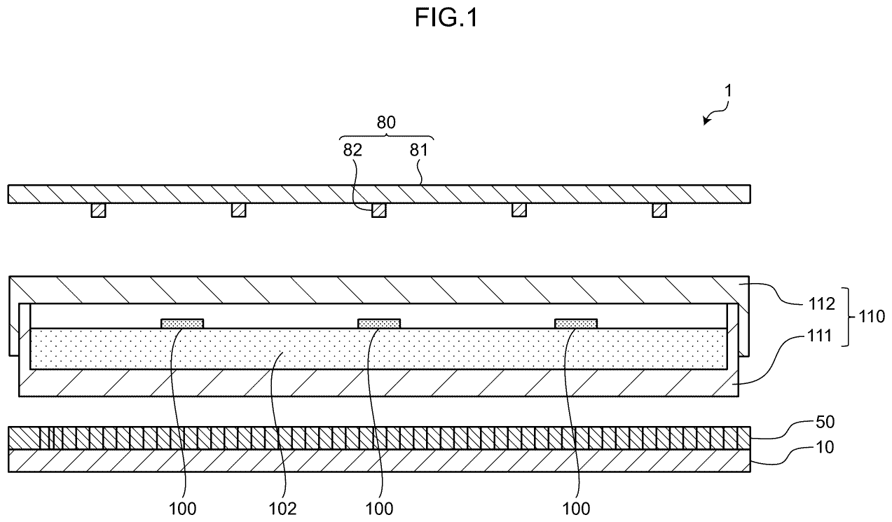

FIG. 1 is a sectional view schematically illustrating a detection device according to an embodiment. As illustrated in FIG. 1, a detection device 1 includes an optical sensor 10, an optical filter layer 50, a container 110 for accommodating an object to be detected 100, and a light source 80. The container 110 (object to be detected 100) is placed between the optical sensor 10 and the light source 80. In the present embodiment, the optical sensor 10, the optical filter layer 50, the container 110 (object to be detected 100), and the light source 80 are arranged in this order in the detection device 1. However, the order of the arrangement is not limited to this order. The light source 80, the container 110 (object to be detected 100), the optical filter layer 50, and the optical sensor 10 may be arranged in this order in the detection device 1.

The object to be detected 100 is, for example, a micro-object such as a bacterium. The bacteria or the like that have been cultured on a culture medium 102 (e.g., agar) and grown into a clump large enough to be visible may be referred to as a colony. The detection device 1 is a biosensor that detect the micro-object such as the bacterium. The object to be detected 100 is not limited to the bacterium, but may be another micro-object such as a cell.

The container 110 includes a container body 111 and a cover member 112. The container 110 is a Petri dish, for example. The container 110 is light-transmitting. The container body 111 accommodates the culture medium 102, and the object to be detected 100 is cultured on the culture medium 102. That is, the container 110 (at least the container body 111, of the container body 111 and the cover member 112) is an object placement member that has a light-transmitting property and in which (on which) a plurality of the objects to be detected 100 can be placed.

In the present embodiment, the container 110 is placed such that the container body 111 is located on the lower side and the cover member 112 is located on the upper side. The container 110 is not limited to this placement, and may be placed upside down. That is, the container 110 may be placed such that the container body 111 is located on the upper side and the cover member 112 is located on the lower side. In this case, the objects to be detected 100 such as the bacteria are placed on the upper side of the culture medium 102 and cultured, and when imaging the objects to be detected 100, the container 110 is placed upside down such that the objects to be detected 100 is positioned on the lower side of the culture medium 102. The objects to be detected 100 serving as a detection target and the culture medium 102 are contained in the container 110 and positioned between the optical sensor 10 and the light source 80.

The optical sensor 10 is a detection device including a plurality of photodiodes 30 arranged in a planar configuration. Each of the photodiodes 30 is a photodetection element that outputs an electrical signal corresponding to light emitted thereto. More specifically, the photodiode 30 is a positive-intrinsic-negative (PIN) photodiode using an inorganic semiconductor or an organic photodiode (OPD) using an organic semiconductor.

The optical filter layer 50 is a light directivity control element disposed between a plurality of light-emitting elements 82 (light source 80) and the photodiodes 30 (optical sensor 10). More specifically, the optical filter layer 50 is provided between the photodiodes 30 of the optical sensor 10 and the container 110. The optical filter layer 50 is disposed so as to face the photodiodes 30 of the optical sensor 10. The optical filter layer 50 is an optical element that transmits, toward the photodiodes 30, components of light emitted from the light-emitting elements 82 and traveling in a direction orthogonal to the optical sensor 10. The optical filter layer 50 is also called collimating apertures or a collimator.

The light source 80 includes a light source board 81 and the light-emitting elements 82. The light-emitting elements 82 are point light sources provided correspondingly to the photodiodes 30 of the optical sensor 10. The light-emitting elements 82 are provided on the light source board 81 and arranged so as to face the photodiodes 30 of the optical sensor 10. Each of the light-emitting elements 82 is configured as a light-emitting diode (LED), for example.

The light emitted from the light-emitting elements 82 passes through the cover member 112, the culture medium 102, the container body 111, and the optical filter layer 50, and is emitted toward the photodiodes 30 of the optical sensor 10. The quantity of the light irradiating the photodiodes 30 differs between an area overlapping the objects to be detected 100 and an area not overlapping the objects to be detected 100. As a result, the optical sensor 10 can image the objects to be detected 100.

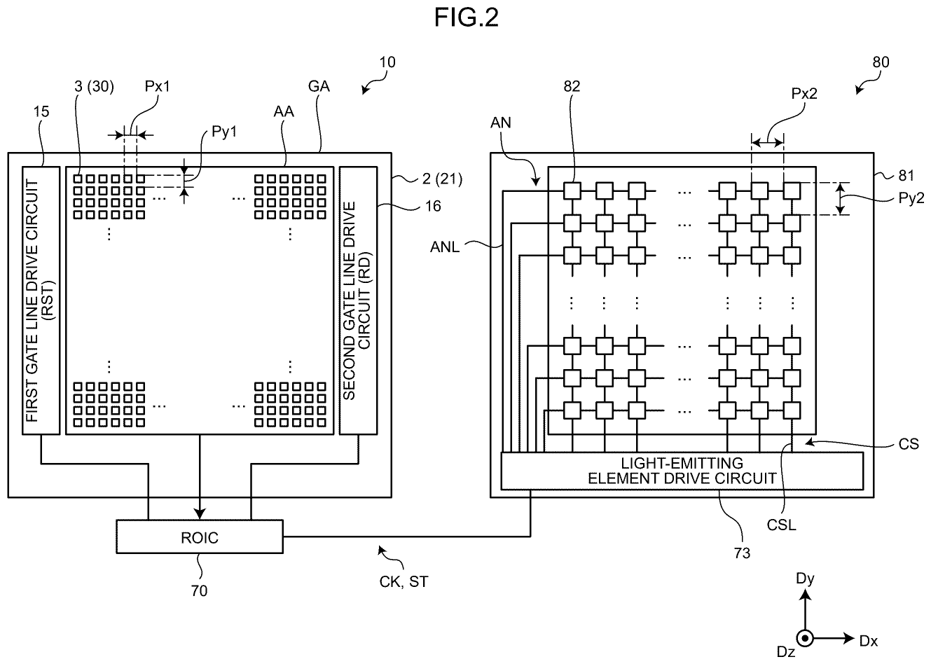

FIG. 2 is a block diagram illustrating a configuration example of the detection device according to the embodiment. As illustrated in FIG. 2, the detection device 1 further includes a control circuit (ROIC) 70 that controls the optical sensor 10 and the light source 80. The control circuit 70 synchronously (or non-synchronously) controls an operation of detecting the objects to be detected 100 with the optical sensor 10 and an operation of lighting the light-emitting elements 82 with the light source 80. The control circuit 70 includes, for example, a microcontroller unit (MCU), a random-access memory (RAM), an electrically erasable programmable read-only memory (EEPROM), and a read-only memory (ROM).

The optical sensor 10 includes an array substrate 2, a plurality of sensor pixels 3 (photodiodes 30) provided on the array substrate 2, a first gate line drive circuit 15, and a second gate line drive circuit 16.

The array substrate 2 is formed using a substrate 21 as a base. Each of the sensor pixels 3 is configured with the photodiode 30, a plurality of transistors, and various types of wiring. The array substrate 2 with the photodiodes 30 formed thereon is a drive circuit board for driving the sensor for each predetermined detection area and is also called a backplane or an active-matrix substrate.

The substrate 21 has a detection area AA and a peripheral area GA. The sensor pixels 3 (photodiodes 30) are arranged in a matrix having a row-column configuration in the detection area AA. That is, the photodiodes 30 are arranged in a first direction Dx and a second direction Dy intersecting the first direction Dx. The first gate line drive circuit 15 and the second gate line drive circuit 16 are provided in the peripheral area GA.

In the following description, the first direction Dx is one direction in a plane parallel to the substrate 21. The second direction Dy is one direction in the plane parallel to the substrate 21 and is a direction orthogonal to the first direction Dx. The second direction Dy may non-orthogonally intersect the first direction Dx. A third direction Dz is a direction orthogonal to the first direction Dx and the second direction Dy and is a direction normal to a principal surface of the substrate 21. The term “plan view” refers to a positional relation when viewed in a direction orthogonal to the substrate 21.

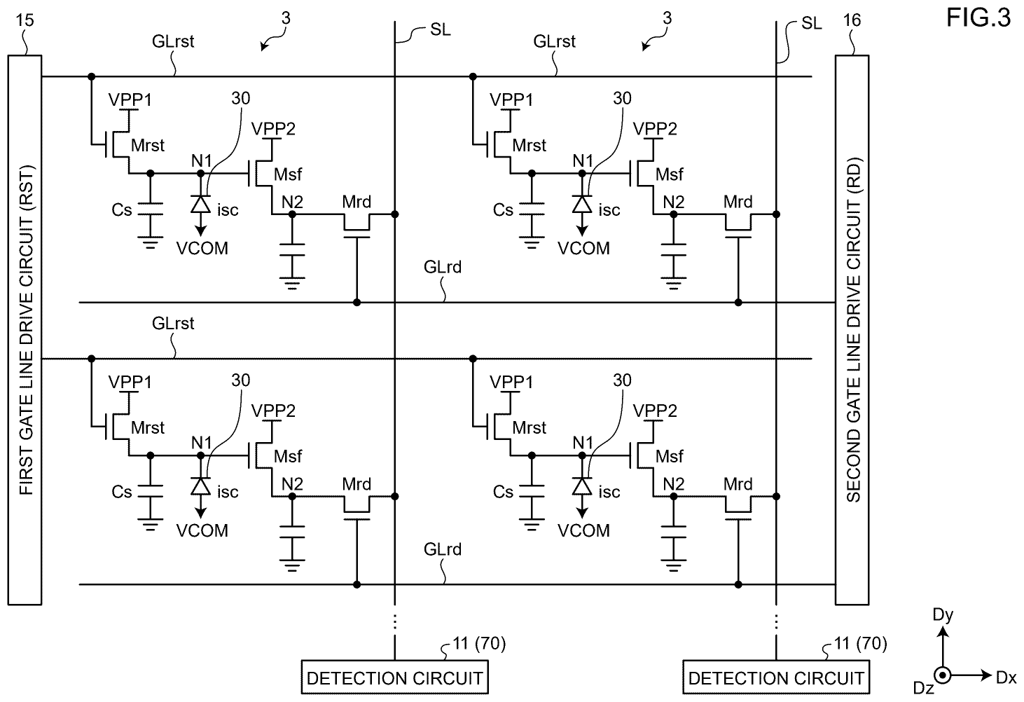

The control circuit 70 is a circuit that supplies respective control signals (clock signals CLK, start signals ST, and other signals) to the first gate line drive circuit 15 and the second gate line drive circuit 16 to control operations of these circuits. Specifically, the first gate line drive circuit 15 outputs a gate drive signal (for example, reset control signal RST) to a reset control scan line GLrst (refer to FIG. 3) based on a control signal. The second gate line drive circuit 16 outputs a gate drive signal (for example, readout control signal RD) to a readout control scan line GLrd (refer to FIG. 3) based on a control signal. The control circuit 70 may be provided on a wiring board electrically coupled to the array substrate 2 or provided in the peripheral area GA of the array substrate 2.

The photodiodes 30 included in the sensor pixels 3 perform detection in response to the gate drive signals supplied from the first gate line drive circuit 15 and the second gate line drive circuit 16. Each of the photodiodes 30 outputs the electrical signal corresponding to the light irradiating the photodiode 30 as a detection voltage Vdet to a detection circuit 11 (refer to FIG. 3). The detection circuit 11 performs signal processing on the detection voltages Vdet from the photodiodes 30 and outputs sensor values So based on the detection voltages Vdet to the control circuit 70. Thus, the detection device 1 detects information on the objects to be detected 100. The detection circuit 11 may be included in the control circuit 70 or may be provided as a circuit different from the control circuit 70.

The light source 80 includes a light-emitting element drive circuit 73 that drives the light-emitting elements 82 mounted on the light source board 81. The light-emitting elements 82 are arranged in a matrix having a row-column configuration in an area of the light source board 81 overlapping the detection area AA. That is, the light-emitting elements 82 are arranged in the first direction Dx and the second direction Dy intersecting the first direction Dx. The light-emitting element drive circuit 73 supplies power supply voltages (anode power supply voltage AN and cathode power supply voltage CS) to the light-emitting elements 82 based on the control signals (clock signals CLK, start signals ST, and other signals) from the control circuit 70. This operation switches the light-emitting elements 82 between on (lit state) and off (unlit state).

The anodes of the light-emitting elements 82 are supplied with the anode power supply voltage AN through anode power supply lines ANL. The anode power supply lines ANL extend in the first direction Dx and are arranged in the second direction Dy. That is, the light-emitting elements 82 arranged in the first direction Dx are coupled to the same anode power supply line ANL.

The cathodes of the light-emitting elements 82 are supplied with the cathode power supply voltage CS through cathode power supply lines CSL. The cathode power supply lines CSL extend in the second direction Dy and are arranged in the first direction Dx. That is, the light-emitting elements 82 arranged in the second direction Dy are coupled to the same cathode power supply line CSL.

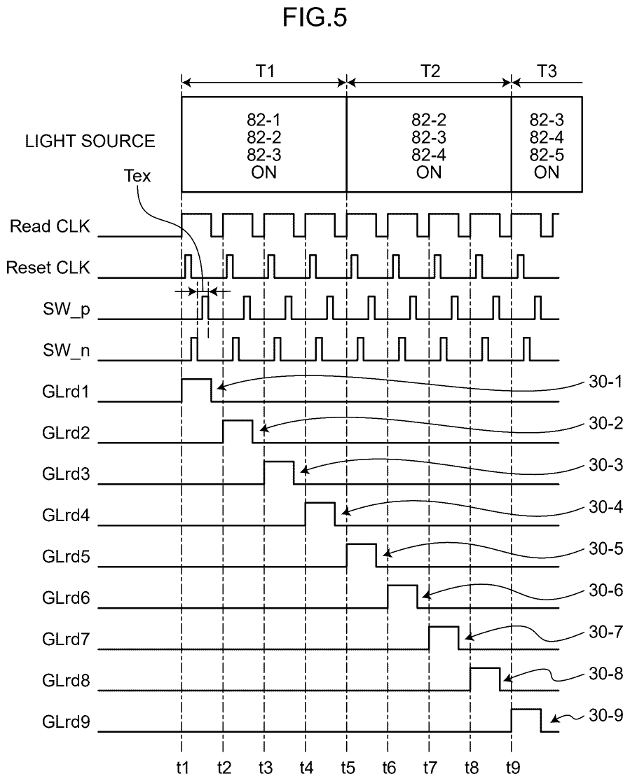

The light-emitting element drive circuit 73 sequentially supplies the anode power supply voltage AN to the anode power supply lines ANL in a time-division manner based on the control signals from the control circuit 70. The light-emitting element drive circuit 73 simultaneously supplies the cathode power supply voltage CS to the cathode power supply lines CSL based on the control signals from the control circuit 70. As a result, the light-emitting elements 82 are sequentially driven along the second direction Dy at least one row by one row. A method for driving the light-emitting elements 82 will be described later in detail with reference to FIG. 5 and the subsequent drawings. The wiring patterns of the anode power supply lines ANL and the cathode power supply lines CSL for driving the light-emitting elements 82 are merely exemplary, and any configuration may be employed as long as the light-emitting elements 82 can be sequentially driven at least one row by one row. For example, the light source board 81 of the light source 80 may be configured as an active-matrix substrate.

The number of the light-emitting elements 82 is smaller than the number of the photodiodes 30. An arrangement pitch Px2 in the first direction Dx of the light-emitting elements 82 is larger than an arrangement pitch Px1 in the first direction Dx of the photodiodes 30. An arrangement pitch Py2 in the second direction Dy of the light-emitting elements 82 is larger than an arrangement pitch Py1 in the second direction Dy of the photodiodes 30.

As illustrated in FIG. 2, each of the arrangement pitches Px1, Py1, Px2, and Py2 is an arrangement interval of one side of the outer shape of the photodiode 30 or the light-emitting element 82. However, the arrangement pitches Px1, Py1, Px2, and Py2 are not limited to these intervals and may be intervals between respective geometric centers of the light-emitting elements 82 and the photodiodes 30.

The following describes a circuit configuration and an operation example of the optical sensor 10. FIG. 3 is a circuit diagram illustrating the optical sensor of the detection device according to the embodiment. As illustrated in FIG. 3, the sensor pixel 3 includes the photodiode 30, a reset transistor Mrst, a readout transistor Mrd, and a source follower transistor Msf. The sensor pixel 3 is provided with the reset control scan line GLrst and the readout control scan line GLrd as detection drive lines (gate lines) and provided with a signal line SL as wiring for signal reading.

The reset control scan line GLrst, the readout control scan line GLrd, and the signal line SL are each coupled to the sensor pixels 3. Specifically, the reset control scan line GLrst and the readout control scan line GLrd extend in the first direction Dx and are coupled to the sensor pixels 3 arranged in the first direction Dx. The signal line SL extends in the second direction Dy and is coupled to the sensor pixels 3 arranged in the second direction Dy. The signal line SL is wiring through which signals from the transistors (readout transistor Mrd and source follower transistor Msf) are output.

The reset transistor Mrst, the readout transistor Mrd, and the source follower transistor Msf are provided correspondingly to one photodiode 30. The transistors included in the sensor pixel 3 are each configured as an n-type thin-film transistor (TFT). However, each of the transistors is not limited thereto and may be configured as a p-type TFT.

A common voltage VCOM is applied to the anode of the photodiode 30. The cathode of the photodiode 30 is coupled to a node N1. The node N1 is coupled to the gate of the source follower transistor Msf and one of the source and the drain of the reset transistor Mrst. When the light irradiates the photodiode 30, a signal (electric charge) output from the photodiode 30 is stored in a capacitive element Cs formed at the node N1.

The gate of the reset transistor Mrst is coupled to the reset control scan line GLrst. The other of the source and the drain of the reset transistor Mrst is supplied with a reset voltage VPP1. When the reset transistor Mrst is turned on (conducting state) in response to the reset control signal RST supplied from the first gate line drive circuit 15, the voltage of the node N1 is reset to the reset voltage VPP1. The common voltage VCOM has a voltage lower than the reset voltage VPP1, and the photodiode 30 is driven in a reverse bias state.

The source follower transistor Msf is coupled between a terminal supplied with a power supply potential VPP2 and the readout transistor Mrd (node N2). The gate of the source follower transistor Msf is coupled to the node N1. The gate of the source follower transistor Msf is supplied with a signal (voltage) corresponding to the signal (electric charge) generated by the photodiode 30. Thus, the source follower transistor Msf outputs a voltage corresponding to the signal (electric charge) generated by the photodiode 30 to the readout transistor Mrd.

The readout transistor Mrd is coupled between the source of the source follower transistor Msf (node N2) and the signal line SL. The gate of the readout transistor Mrd is coupled to the readout control scan line GLrd. When the readout transistor Mrd is turned on in response to the readout control signal RD supplied from the second gate line drive circuit 16, the signal output from the source follower transistor Msf, that is, the signal (voltage) corresponding to the signal (electric charge) generated by the photodiode 30 is output as the detection voltage Vdet to the signal line SL. The signal lines SL are each coupled to the detection circuit 11.

In FIG. 3, the reset transistor Mrst and the readout transistor Mrd each have a single-gate structure. However, the reset transistor Mrst and the readout transistor Mrd may each have a double-gate structure composed of two transistors coupled in series or may be have a configuration composed of three or more transistors coupled in series. The circuit of one sensor pixel 3 is not limited to the configuration including the three transistors of the reset transistor Mrst, the source follower transistor Msf, and the readout transistor Mrd. The sensor pixel 3 may include two transistors or four or more transistors.

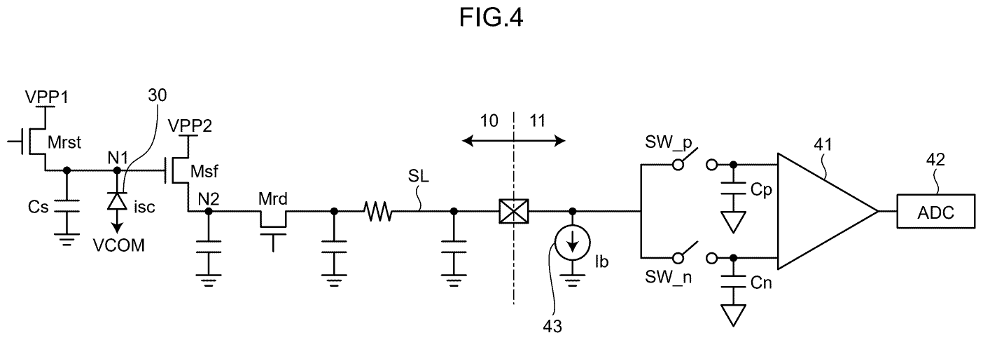

FIG. 4 is a circuit diagram illustrating a configuration example of the sensor pixel and the detection circuit of the detection device according to the embodiment. To facilitate understanding of the description, FIG. 4 illustrates the example in which one sensor pixel 3 (photodiode 30) is coupled to one detection circuit 11.

As illustrated in FIG. 4, the detection circuit 11 includes an amplifying circuit 41, an analog-to-digital (A/D) conversion circuit 42, a first switch element SW_p, a first capacitive element Cp, a second switch element SW_n, and a second capacitive element Cn. The first switch element SW_p and the first capacitive element Cp are coupled to the non-inverting input (+) of the amplifying circuit 41. The second switch element SW_n and the second capacitive element Cn are coupled to the inverting input (−) of the amplifying circuit 41.

One end of the first switch element SW_p is electrically coupled to the output of the readout transistor Mrd via the signal line SL. The other end of the first switch element SW_p is coupled to the first capacitive element Cp and the non-inverting input (+) of the amplifying circuit 41.

One end of the second switch element SW_n is electrically coupled to the output of the readout transistor Mrd via the signal line SL. The other end of the second switch element SW_n is coupled to the second capacitive element Cn and the inverting input (−) of the amplifying circuit 41. The detection circuit 11 switches the coupling states of the first switch element SW_p and the second switch element SW_n in synchronization with the control signals from the control circuit 70. This operation electrically couples the signal line SL to one of the non-inverting input (+) and the inverting input (−) of the amplifying circuit 41.

During a reset period, the first gate line drive circuit 15 sets the reset control scan line GLrst to a high-level voltage (“H”), and the second gate line drive circuit 16 sets the readout control scan line GLrd to “H”. These operations turn on the reset transistor Mrst and the readout transistor Mrd. During the reset period, the detection circuit 11 controls the first switch element SW_p such that the first switch element SW_p is off and controls the second switch element SW_n such that the second switch element SW_n is on. These operations charge the second capacitive element Cn with an electric charge corresponding to the reset voltage VPP1 during the reset period.

During an exposure period after the reset period, the light is emitted from the light source 80 (light-emitting elements 82) to the photodiode 30. During a readout period after the exposure period, the first gate line drive circuit 15 sets the reset control scan line GLrst to a low-level voltage (“L”), and the second gate line drive circuit 16 sets the readout control scan line GLrd to “H”. These operations turn off the reset transistor Mrst and turn on the readout transistor Mrd. During the readout period, the detection circuit 11 controls the first switch element SW_p such that the first switch element SW_p is on and controls the second switch element SW_n such that the second switch element SW_n is off. These operations charge the first capacitive element Cp with an electric charge corresponding to the detection voltage Vdet (voltage of the node N2 after the exposure).

The amplifying circuit 41 amplifies the potential difference between the reset voltage VPP1 charged in the second capacitive element Cn during the reset period and the detection voltage Vdet charged in the first capacitive element Cp during the readout period. The A/D conversion circuit 42 converts the value amplified by the amplifying circuit 41 into a digital signal.

Thus, in the detection device 1 of the present embodiment, a sampling time to acquire the digital data corresponding to each of the sensor pixels 3 is provided for each “H” period of the readout control scan line GLrdn in the n-th row (that is, for each n-th row of the photodiodes 30-n to be driven).

The detection circuit 11 is coupled to a constant current source 43 to apply a bias current Ib to the readout transistor Mrd. This configuration allows the sensor pixel 3 to detect voltages (reset voltage VPP1 in the reset period and detection voltage Vdet in the readout period). This constant current source 43 may be provided in the detection circuit 11 or in the substrate 21.

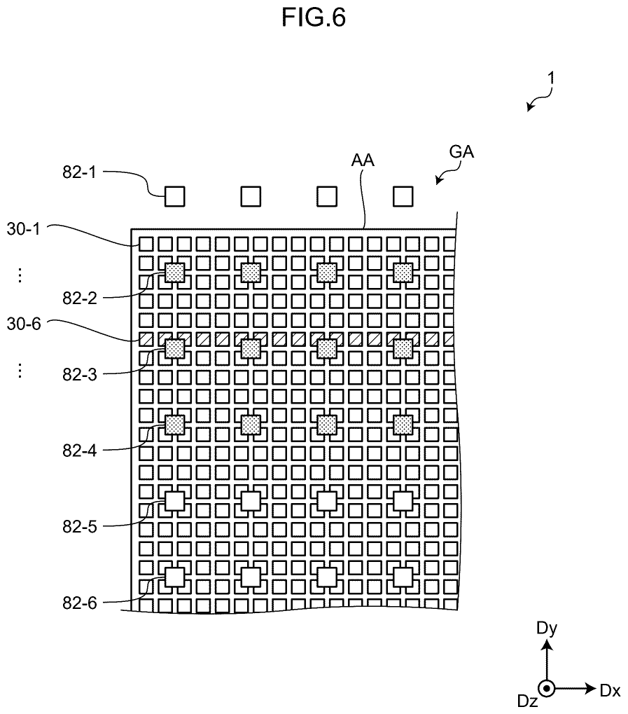

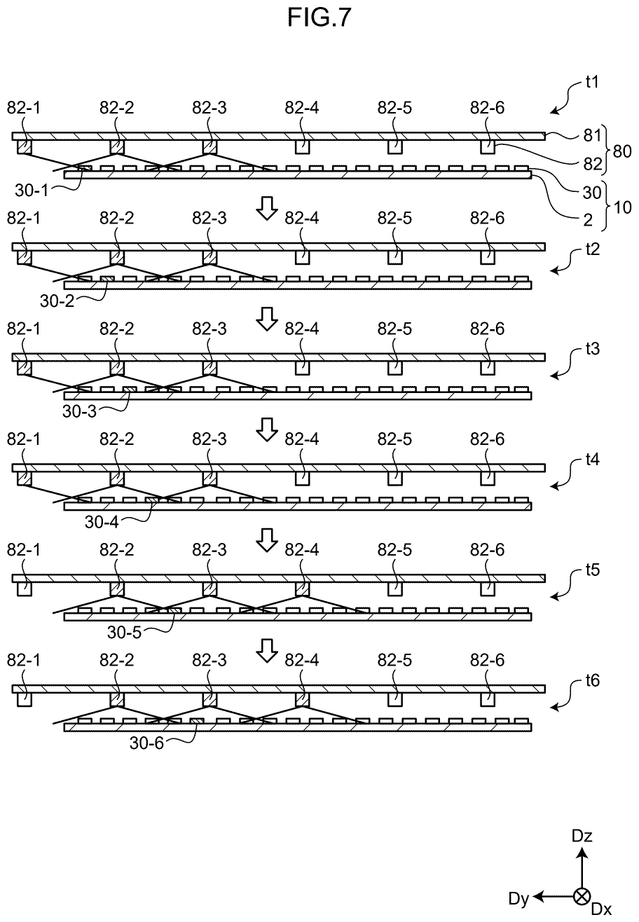

The following describes an operation example of the optical sensor 10 and the light source 80 in the detection device 1 of the present embodiment, with reference to FIGS. 5 to 7. FIG. 5 is a timing waveform diagram illustrating the operation example of the detection device according to the embodiment. FIG. 6 is a plan view schematically illustrating the operation example of the detection device according to the embodiment. FIG. 7 is a sectional view schematically illustrating the operation example of the detection device according to the embodiment.

In FIGS. 6 and 7, the photodiodes 30 that are driven are illustrated with hatching, and the photodiodes 30 that are not driven are illustrated without hatching. The photodiodes 30 that are driven are the photodiodes 30 in the sensor pixels 3 for which the readout control scan line GLrd is set to “H”. The photodiodes 30 that are not driven are the photodiodes 30 in the sensor pixels 3 for which the readout control scan line GLrd is set to “L”.

In FIGS. 6 and 7, the light-emitting elements 82 that are lit are illustrated with hatching, and the light-emitting elements 82 that are not lit are illustrated without hatching. The light-emitting elements 82 that are lit are supplied with the anode power supply voltage AN from the light-emitting element drive circuit 73. The light-emitting elements 82 that are not lit are not supplied with the anode power supply voltage AN from the light-emitting element drive circuit 73.

FIG. 6 illustrates the driving of the photodiodes 30 and the lighting of the light-emitting elements 82 corresponding to time t6 in FIGS. 5 and 7.

As illustrated in FIGS. 5 to 7, the photodiodes 30 are sequentially driven row by row along the second direction Dy. The light-emitting elements 82 are sequentially driven in units of three rows of the light-emitting elements 82 along the second direction Dy. At a given time, the photodiodes 30 to be driven among the photodiodes 30 correspond to the light-emitting elements 82 to be lit among the light-emitting elements 82 in plan view.

More specifically, as illustrated in FIG. 5, at time t1, the second gate line drive circuit 16 sets a readout control scan line GLrd1 in the first row to “H” based on a readout clock signal ReadCLK. This operation turns on the readout transistors Mrd coupled to the readout control scan line GLrd1 in the first row and drives photodiodes 30-1 in the first row. The photodiodes 30 in the other rows other than the driven photodiodes 30-1 in the first row are not driven.

In the period during which the readout control scan line GLrd1 in the first row is at “H”, the first gate line drive circuit 15 sets the reset control scan line GLrst to “H” (not illustrated in FIG. 5) based on a reset clock signal ResetCLK. This operation resets the photodiodes 30-1 in the first row. After the elapse of a predetermined period of time, the reset control scan line GLrst is set to “L” based on the reset clock signal ResetCLK, and the reset period of the photodiodes 30-1 in the first row ends.

After the end of the reset period, the detection circuit 11 turns on the second switch element SW_n in the period during which the readout control scan line GLrd in the first row is at “H”. This operation charges the second capacitive element Cn with the electric charge corresponding to the reset voltage VPP1. After that, the second switch element SW_n is turned off. After the elapse of a predetermined period of time since the second switch element SW_n is turned off, the detection circuit 11 turns on the first switch element SW_p. This operation charges the first capacitive element Cp with the electric charge corresponding to the detection voltage Vdet (voltage of the node N2 after the exposure) of the photodiode 30.

In the present embodiment, the period during which the first switch element SW_p is on is the readout period of the driven photodiodes 30-1 in the first row. A period from when the second switch element SW_n is turned off to when the first switch element SW_p is turned off is a substantial exposure period Tex of the driven photodiodes 30-1 in the first row. The substantial exposure period Tex refers to a period during which the output value of the amplifier is affected even if the light source 80 is lit before and/or after this period.

The first switch element SW_p is turned off, and the readout period and the exposure period Tex of the photodiodes 30-1 in the first row end. After the readout period and the exposure period Tex end, the second gate line drive circuit 16 sets the readout control scan line GLrd1 in the first row to “L” based on the readout clock signal ReadCLK.

Then, at time t2, the second gate line drive circuit 16 sets a readout control scan line GLrd2 in the second row to “H” based on the readout clock signal ReadCLK. This operation drives photodiodes 30-2 in the second row. In the same way as the driving of the photodiodes 30-1 in the first row described above, the operations of the reset period, the exposure period Tex, and the readout period of the photodiodes 30-2 in the second row are performed during the period in which the readout control scan line GLrd2 is at “H”.

Subsequently, at time t3 and later, the second gate line drive circuit 16 sequentially sets the readout control scan line GLrdn in the n-th row (n is a natural number) to “H” based on the readout clock signal ReadCLK. This operation drives the photodiodes 30-n in the n-th row. The operations of the reset period, the exposure period Tex, and the readout period described above are sequentially performed for each n-th row of the photodiodes 30-n to be driven.

In the detection device 1 of the present embodiment, the exposure period Tex is provided for each n-th row of the photodiodes 30-n to be driven as illustrated in FIG. 5. That is, the reset period (period during which the second switch element SW_n is on), the exposure period Tex, and the readout period (during which the first switch element SW_p is on) are provided for each n-th row of the photodiodes 30-n such that those periods are repeatedly provided. The length of the exposure period Tex of the photodiode 30-n in the n-th row is shorter than a period during which the readout control scan line GLrdn in the n-th row is at “H”, that is, a period during which the readout transistor Mrd in the n-th row is on.

As a result, the detection device 1 of the present embodiment can reduce the variation of the exposure period Tex between the rows of the photodiodes 30, compared with, for example, a case where the detection for one frame (readout period) is performed by scanning the photodiodes 30 after performing the reset operation on all the photodiodes 30 in the detection area AA.

As illustrated in FIGS. 6 and 7, the light source 80 turns on the light-emitting elements 82 corresponding to the photodiodes 30 to be driven. The term “light-emitting elements 82 corresponding to” herein refers to the light-emitting elements 82 in a row with the shortest distances in the second direction Dy from the photodiodes 30 to be driven. The term “light-emitting elements 82 corresponding to” (that is, the light-emitting elements 82 to be lit) includes the light-emitting elements 82 arranged overlapping the photodiodes 30 to be driven (for example, refer to times t2, t3, and t6) and the light-emitting elements 82 arranged closer than the other light-emitting elements 82 to the photodiodes 30 to be driven but not overlapping the photodiodes 30 to be driven (for example, refer to times t1, t4, and t5).

Furthermore, the light source 80 turns on the light-emitting elements 82 in two rows that are adjacent to the light-emitting elements 82 corresponding to the photodiode 30 to be driven, wherein one of the two rows is located on one side in the second direction Dy of the light-emitting elements 82 corresponding to the photodiode 30 to be driven, and the other one of the two rows is located on the other side in the second direction of the light-emitting elements 82 corresponding to the photodiode 30 to be driven.

Specifically, in the example illustrated in FIG. 7, at time t1, light-emitting elements 82-2 in the second row are controlled to be in the lit state (on) correspondingly to the photodiodes 30-1 in the first row to be driven. In addition, the light source 80 simultaneously controls light-emitting elements 82-1 and light-emitting elements 82-3 to be in the lit state. The light-emitting elements 82-1 in the first row are adjacent to the light-emitting elements 82-2 and disposed on one side in the second direction Dy of the light-emitting elements 82-2, and the light-emitting elements 82-3 in the third row are adjacent to the light-emitting elements 82-2 and disposed on the other side in the second direction Dy of the light-emitting elements 82-2. At time t1, the light-emitting elements 82 in rows other than the rows of the light-emitting elements 82-1, 82-2, and 82-3 to be lit, are not lit.

At times t2, t3, and t4, the photodiodes 30-2 in the second row, photodiodes 30-3 in the third row, and photodiodes 30-4 in the fourth row are sequentially driven. In this case, the light-emitting elements 82 that correspond to all the photodiodes 30-2, 30-3, and 30-4 to be driven are the light-emitting elements 82-2 in the second row. At times t2, t3, and t4, the light-emitting elements 82-1 and 82-3 in two rows adjacent in the second direction Dy to the light-emitting element 82-2 in the second row are also simultaneously controlled to be in the lit state. That is, also, at times t2, t3, and t4, the light-emitting elements 82-1, 82-2, and 82-3 are controlled to be in the lit state, and the light-emitting elements 82 in the other rows are controlled to be in the unlit state, in the same way as at time t1.

At time t5, photodiodes 30-5 in the fifth row are driven. The light-emitting elements 82-3 in the third row are controlled to be in the lit state correspondingly to the photodiodes 30-5 in the fifth row to be driven. In addition, the light source 80 also simultaneously controls the light-emitting elements 82-2 and the light-emitting elements 82-4 to be in the lit state. The light-emitting elements 82-2 in the second row are adjacent to the light-emitting elements 82-3 in the third row and disposed on one side in the second direction Dy of the light-emitting elements 82-3, and the light-emitting elements 82-4 in the fourth row are adjacent to the light-emitting elements 82-3 in the third row and disposed on the other side in the second direction Dy of the light-emitting elements 82-3. At time t5, the light-emitting elements 82-2, 82-3, and 82-4 in three rows are controlled to be in the lit state, and the light-emitting elements 82 in the other rows are controlled to be in the unlit state.

In other words, at time t5, the positional relation between the photodiodes 30-5 and the light-emitting elements 82 that are driven differs from the positional relation from time t1 to time t4. As a result, the light-emitting elements 82 in three rows to be lit are shifted by one row in the second direction Dy. At time t4, the photodiodes 30-4 are driven, and the light-emitting elements 82-2 in the second row and the light-emitting elements 82-3 in the third row are in the lit state. The light-emitting elements 82-2 in the second row (light-emitting elements 82 in the first row) have the shortest distances in the second direction Dy from the photodiodes 30-4, and the light-emitting elements 82-3 in the third row (light-emitting elements 82 in the second row) are adjacent to the light-emitting elements 82-2 and disposed on the other side in the second direction Dy of the light-emitting elements 82-2. At the next time t5, when the photodiodes 30-5 closer to the light-emitting elements 82-3 as compared with the photodiodes 30-4 are driven, the light-emitting element 82-4 are controlled to be in the lit state (turned on).

As illustrated in FIGS. 6 and 7, at time t6, photodiodes 30-6 in the sixth row are driven. Correspondingly to the photodiodes 30-6 to be driven, the light-emitting elements 82-3 in the third row with the shortest distances in the second direction Dy from the photodiodes 30-6 are in the lit state. That is, at time t6, the light-emitting elements 82 that correspond to the photodiodes 30-6 to be driven are the light-emitting elements 82-3 in the third row. In addition, the light-emitting elements 82-2 in the second row and the light-emitting elements 82-4 in the fourth row are also simultaneously controlled to be in the lit state. The light-emitting elements 82-2 in the second row and the light-emitting elements 82-4 in the fourth row are adjacent in the second direction Dy to the light-emitting elements 82-3 in the third row. At time t6, the light-emitting elements 82-2, 82-3, and 82-4 in the three rows are controlled to be in the lit state, and the light-emitting elements 82 in the other rows are controlled to be in the unlit state.

Hereafter, in the same way, during periods when photodiodes 30-7 and photodiodes 30-8 in the seventh and eighth rows, respectively, are driven, the light-emitting elements 82-2, 82-3, and 82-4 in the three rows are controlled to be in the lit state (on), and the light-emitting elements 82 in the other rows are controlled to be in the unlit state (off), as illustrated in FIG. 5. At time t9, when photodiodes 30-9 in the ninth row are driven, the light source 80 shifts the light-emitting elements 82 to be lit. As a result, the light-emitting elements 82-3, 82-4, and 82-5 in three rows are controlled to be in the lit state (on), and the light-emitting elements 82 in the other rows are controlled to be in the unlit state (off). Thus, the photodiodes 30 and the light-emitting elements 82 are sequentially driven row by row.

As described above, in the detection device 1 of the present embodiment, light-emitting elements 82-m in the m-th row corresponding to the photodiodes 30-n in the n-th row to be driven are controlled to be in the lit state. In the present embodiment, at least the light-emitting elements 82-m are turned on, and the light-emitting elements 82 in the other rows that differ from the light-emitting elements 82-m to be lit are controlled to be unlit. In the example illustrated in FIGS. 5 to 7, the light-emitting elements 82-m and light-emitting elements 82-(m−1) and 82-(m+1) adjacent to the light-emitting elements 82-m are controlled to be in the lit state, and the light-emitting elements 82 in the other rows are controlled to be in the unlit state. Therefore, the detection device 1 can reduce the power loss of the light source 80 compared with a case where the light-emitting elements 82 in the entire area are lit.

In addition, the light source 80 also controls the light-emitting elements 82-(m−1) and 82-(m+1) to be in the lit state. The light-emitting elements 82-(m−1) and 82-(m+1) are adjacent to the light-emitting elements 82-m in the m-th row (light-emitting elements 82 corresponding to the photodiodes 30-n in the n-th row to be driven). As a result, even if the distances between the photodiodes 30-n in the n-th row to be driven and the light-emitting elements 82-m in the m-th row to be lit are different, the light is emitted from the light-emitting elements 82 to the photodiodes 30-n in the n-th row. Therefore, the variation of the light quantity can be reduced.

Specifically, as illustrated in FIG. 7, the distance in the second direction Dy between the photodiode 30-1 in the first row to be driven and the corresponding light-emitting element 82-2 in the second row at time t1 is larger than the distance in the second direction Dy between the photodiode 30-2 in the second row to be driven and the corresponding light-emitting element 82-2 in the second row at time t2. Even in this case, the photodiode 30-1 in the first row driven at time t1 is irradiated with light from the light-emitting element 82-2 in the second row and the light-emitting element 82-1 in the first row adjacent thereto. As a result, the variation between the quantity of the light irradiating the photodiode 30-1 in the first row and the quantity of the light irradiating the photodiode 30-2 in the second row can be reduced.

The operation example illustrated in FIGS. 5 to 7 is merely exemplary and can be changed as appropriate. The photodiodes 30 are not limited to the example of being sequentially driven row by row, and may be driven in units of a plurality of rows depending on the required detection accuracy (resolution), the detection period of one frame, or the like. The example has been illustrated where the light-emitting elements 82 in three rows are simultaneously in the lit state for the photodiodes 30 in one row to be driven. The present disclosure is not limited to this example. The light-emitting elements 82 only needs to be driven at least row by row.

While the preferred embodiment of the present disclosure has been described above, the present disclosure is not limited to the embodiment described above. The content disclosed in the embodiment is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiment and modifications described above.

What is claimed is:

1. A detection device comprising:
an optical sensor comprising a plurality of photodiodes arranged in a planar configuration;
a light source comprising a plurality of light-emitting elements configured to emit light to the photodiodes; and
an object placement member that has a light-transmitting property and is configured to be disposed between the optical sensor and the light source, and on which a plurality of objects to be detected are to be placed, wherein
the photodiodes are arranged in a matrix having a row-column configuration in a first direction and a second direction intersecting the first direction and are configured to be sequentially driven along the second direction at least one row by one row,
the light-emitting elements are arranged in a matrix having a row-column configuration in the first direction and the second direction and are configured to be sequentially driven along the second direction at least one row by one row, and
at a given time, photodiodes to be driven among the photodiodes correspond to light-emitting elements to be lit among the light-emitting elements in plan view.

2. The detection device according to claim 1, wherein a row of the light-emitting elements with shortest distances in the second direction from the photodiodes to be driven are configured to be lit.

3. The detection device according to claim 1, wherein two rows of the light-emitting elements that are adjacent to the row of the light-emitting elements corresponding to the photodiodes to be driven are further configured to be lit, one of the two rows is located on one side in the second direction of the row of the photodiodes to be driven and the other one of the two rows is located on the other side in the second direction of the row of the photodiodes to be driven.

4. The detection device according to claim 1, wherein
the number of the light-emitting elements is smaller than the number of the photodiodes, and
when the light-emitting elements in a first row and a second row that are adjacent to each other in the second direction are lit and the photodiodes to be driven are closer to the light-emitting elements in the second row, the light-emitting elements in a third row adjacent to the second row in the second direction are further configured to be lit.

5. The detection device according to claim 1, wherein the light-emitting elements are light-emitting diodes (LEDs).
